(19) United States
(12) Patent Application Publication
(10) Pub. No.: US 2021/0113109 A1
(43) Pub. Date: Apr. 22, 2021

(54) SILICON ENCAPSULATED HEART MONITORING DEVICE

(71) Applicant: Medicomp, Inc., Melbourne, FL (US)

(72) Inventors: Anthony Balda, Melbourne, FL (US); George Koos, Melbourne, FL (US); Michael McClean, Melbourne, FL (US)

(73) Assignee: Medicomp, Inc., Melbourne, FL (US)

(21) Appl. No.: 16/949,263

(22) Filed: Oct. 22, 2020

Related U.S. Application Data
(60) Provisional application No. 62/924,240, filed on Oct. 22, 2019.

(51) Int. Cl. A61B 5/0408; A61B 5/00; A61B 5/0416

(52) U.S. Cl. CPC A61B 5/04087; A61B 5/68335; A61B 2562/227; A61B 2562/166; A61B 5/0416

(57) ABSTRACT

A system for heart monitoring including an electronic monitoring unit, a flexible carrier member, and a liner. The flexible carrier member includes a central void adapted to carry the electronic monitoring unit within the void and a back surface having a first connector component. The liner includes a back, adhesive surface, an opposing front surface, and a second connector component located on the front surface and adapted to secure to the first connector component of the flexible carrier member.

### RELATED APPLICATIONS

This application claims priority under 35 U.S.C. § 119(e) of U.S. Provisional Patent Application Serial No. (Attorney Docket No. 612.00145) filed on Oct. 22, 2019 and titled SILICON ENCAPSULATED HEART MONITORING DEVICE. The content of this application is incorporated herein by reference.

### FIELD OF THE INVENTION

The present invention relates to systems and methods for heart monitoring. More specifically, the present invention is directed to a reusable, body-worn heart monitoring system.

### BACKGROUND OF THE INVENTION

Heart disease is a leading cause of death in the United States. Some patients may benefit from long-term ECG monitoring outside of a clinical setting. For example, atrial fibrillation and myocardial ischemia may occur episodically. Some episodes may occur without patient symptoms. Myocardial ischemia, if persistent and serious, can lead to myocardial infarction (heart attack). During a myocardial infarction, electrophysiological changes may be detected by an ECG monitoring device. For accurate diagnosis and effective treatment of many episodic heart conditions, medical professionals need to receive accurate and timely information regarding the frequency and duration of such episodes.

In conventional long-term ECG monitoring, such as with continuous Holter monitors or event monitors, mounting of the monitor typically involves preparation of the patient's skin to receive the monitoring device. Chest hair may be shaved or clipped from men. The skin is abraded to remove dead skin cells and cleaned. A technician trained in electrode placement applies the electrodes to the skin with an adhesive. Each electrode of such conventional monitors is attached to an insulated wire that is routed some distance across the patient's body to an amplifier designed to amplify the ECG signal in preparation for further processing. Such monitoring systems are often worn by a patient for up to a month.

Traditional long-term monitoring systems like those described above present a number of problems. During use, the patient must be careful not to pull on the wires connected to the electrodes, lest the electrodes be pulled off the skin. Removing an electrode with its strong adhesive may be painful to the patient. Furthermore, certain types of electrodes require use of a gel next to the skin to improve conductivity at the point of connection of the metal electrode to the skin. Prolonged exposure to the gel can irritate the skin. These and other discomfort factors associated with traditional long-term monitoring solutions may discourage a patient from using the ECG monitor as directed by medical personnel.

Alternative health monitoring system designs exist that attempt to address the many shortcomings of traditional ECG wired monitors.

No device currently exists that supports long term wear of a reusable heart monitoring device without significant disposable portions. Consequently, a need exists for increasingly comfortable, convenient, and reusable monitoring devices for both personal and medical use, and that overcome the shortcomings of common implementations in the field.

This background information is provided to reveal information believed by the applicant to be of possible relevance to the present invention. No admission is necessarily intended, nor should be construed, that any of the preceding information constitutes prior art against the present invention. This reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### SUMMARY OF THE INVENTION

With the above in mind, embodiments of the present invention are related to a system for heart monitoring including an electronic monitoring unit, a flexible carrier member, and a liner.

The electronic monitoring unit may include a microphone. The electronic monitoring unit may activate the microphone to capture an audio recording. The audio recording may be associated with a time period of recording activity.

The flexible carrier member may include a central void adapted to carry the electronic monitoring unit within the void and a back surface having a first connector component. The flexible carrier member may further include a front receptacle portion having the central void and a back cover portion. The first connector component may be carried between the front receptacle portion and the back cover portion.

The front receptacle portion of the flexible carrier member may further include a plurality of sidewalls defining the central void, a lead cover portion, and a connector cover. The lead cover portion may extend from one of the plurality of sidewalls distal the central void and have a first end secured to one of the plurality of sidewalls. The connector cover may be secured to a second end of the lead cover portion.

The back cover portion of the flexible carrier member may further include an aperture adapted to carry the first connector component. The aperture, the first connector component, and the second connector component may be in vertical alignment. The back cover portion may further include a connector receptacle adapted to carry the first connector component. The back cover portion may further include an aperture, a connector, and a lead. The aperture may be located through an entirety of a thickness of the front receptacle portion. The connector may be secured between the front receptacle portion and the back cover portion and have at least one contact accessible through the aperture. The lead may be located between the lead cover portion and the back cover portion and have a first end secured to the first connector component and a second end secured to the connector.

The liner may include a back, adhesive surface, an opposing front surface, and a second connector component located on the front surface and adapted to secure to the first connector component of the flexible carrier member. The liner may also include a foam layer having a front and an opposing, rear surface. The front surface of the foam layer may be the front surface of the liner. The liner may include an adhesive layer having a front and an opposing, back surface, wherein the back surface of the adhesive layer is the back, adhesive surface of the liner and the rear surface of the foam layer secures to the front surface of the adhesive layer. The liner may include a securing component positioned on the back surface of the adhesive layer and in electrical communication with the second connector. The second connector may be positioned on the front surface of the foam layer. The liner may include a hydrogel deposit in electrical communication with the securing component. The liner may include a washer captured between the second connector and the foam layer. An outer perimeter of the foam layer may be contained within an outer perimeter of the adhesive layer.

The first connector component may include a socket or stud of a snap fastener and the second connector component includes a socket or stud of a snap fastener.

The second connector may be adapted to be electrical and physical communication with the first connector component.

The system may include an electronic monitoring unit, a flexible carrier member, and a liner.

The flexible carrier member may include a front receptacle portion, a connector aperture, a back cover portion, a connector, a first connector component, a lead cover portion, a connector cover, and a lead. The front receptacle portion may have a plurality of sidewalls defining a central void adapted to carry the electronic monitoring unit within the void. The connector aperture may be located through an entirety of a thickness of the front receptacle portion. The back cover portion may have a back surface. The connector may be secured between the front receptacle portion and the back cover portion and may have at least one contact accessible through the connector aperture. The first connector component may be carried between the front receptacle portion and the back cover portion. The lead cover portion may extend from one of the plurality of sidewalls distal the central void have a first end secured to the one of the plurality of sidewalls. The connector cover may be secured to a second end of the lead cover portion. The lead may be located between the lead cover portion and the back cover portion and have a first end secured to the first connector component and a second end secured to the connector.

The liner may include an adhesive layer, a securing component, a second connector, a foam layer, a hydrogel deposit, and a washer. The adhesive layer may have a front surface, and an opposing back, adhesive surface. The securing component may be positioned on the back surface of the adhesive layer. The foam layer may have a rear surface, secured to the front surface of the adhesive layer, and a front surface. The hydrogel deposit may be in electrical communication with the securing component. The washer may be captured between the second connector and the foam layer.

The second connector may be adapted to physically secure to the first connector component of the flexible carrier member and create an electrical connection therebetween. The securing component may be in electrical communication with the second connector. An outer perimeter of the foam layer may be contained within an outer perimeter of the adhesive layer.

The back cover portion may further include an aperture adapted to carry the first connector component in vertical alignment with the second connector component.

The flexible carrier member may further include a third carrier connector component, a second lead cover portion, a second connector cover, a second lead, a fourth carrier connector, a third lead cover portion, a third connector cover, and a third lead. The third carrier connector component may be carried between the front receptacle portion and the back cover portion. The second lead cover portion may extend from one of the plurality of sidewalls distal the central void and have a first end secured to the one of the plurality of sidewalls. The second connector cover may be secured to a second end of the second lead cover portion and positioned to cover the third carrier connector component. A second lead may be located between the second lead cover portion and the back cover portion and have a first end secured to the third carrier connector component and a second end secured to the connector, A fourth carrier connector component may be carried between the front receptacle portion and the back cover portion. A third lead cover portion may extend from one of the plurality of sidewalls distal the central void have a first end secured to the one of the plurality of sidewalls. The third connector cover may be secured to a second end of the third lead cover portion and positioned to cover the fourth carrier connector component. The third lead may be located between the third lead cover portion and the back cover portion and have a first end secured to the fourth carrier connector component and a second end secured to the connector.

The liner may further include a third liner connector and a fourth liner connector. The third liner connector may be positioned on the front surface of the foam layer. The fourth liner connector may be positioned on the front surface of the foam layer.

The third liner connector may be adapted to physically secure to the third carrier connector component of the flexible carrier member and create an electrical connection therebetween. The fourth liner connector may be adapted to physically secure to the fourth carrier connector component of the flexible carrier member and create an electrical connection therebetween.

### BRIEF DESCRIPTION OF THE DRAWINGS

Some embodiments of the present invention are illustrated as an example and are not limited by the figures of the accompanying drawings, in which like references may indicate similar elements.

FIG. 1 is a top plan view of heart monitor system.

FIG. 2 is a perspective view of the flexible carrier member of the heart monitor system of FIG. 1.

FIG. 3 is a top plan view of the flexible carrier member of FIG. 2.

FIG. 4 is a top plan view of the back cover portion of the flexible carrier member of FIG. 2.

FIG. 5 is a back plan view of the flexible carrier member of FIG. 2.

FIG. 6 is an exploded view of the flexible carrier member of FIG. 2.

FIG. 7 is a perspective view of the liner of the heart monitor system of FIG. 1.

FIG. 8 is a top plan view of the liner of FIG. 7.

FIG. 9 is an exploded view of the liner of FIG. 7.

FIG. 10 is a top plan view of the electronic monitoring unit of the heart monitor system of FIG. 1.

FIG. 11. Is a bottom plan view of the electronic monitoring unit of FIG. 10.

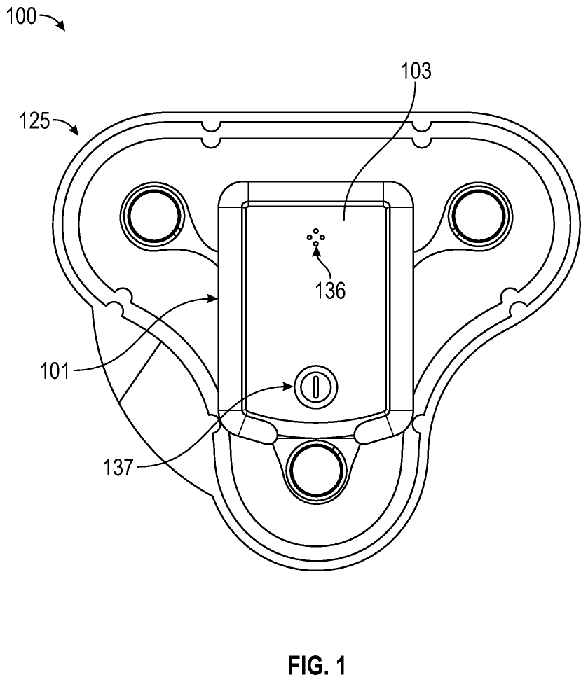

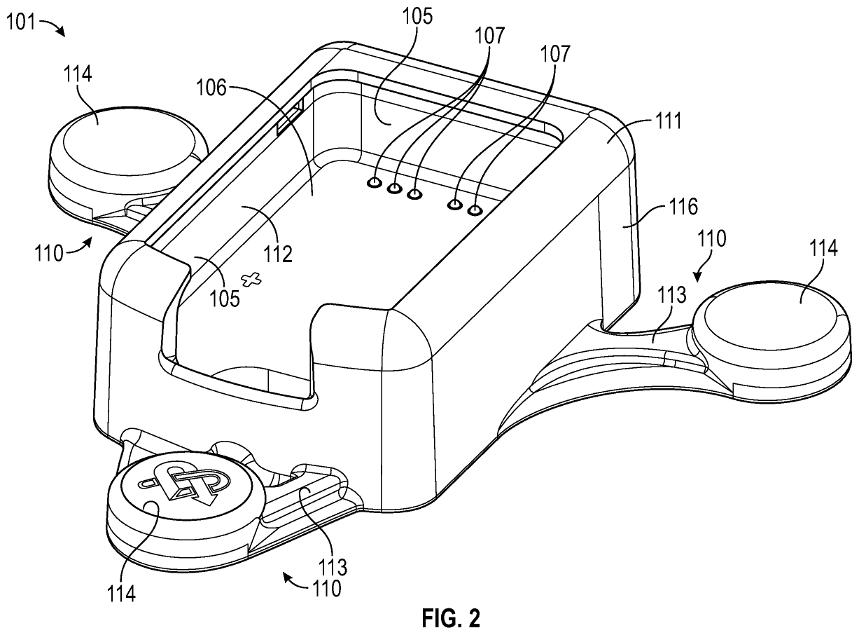

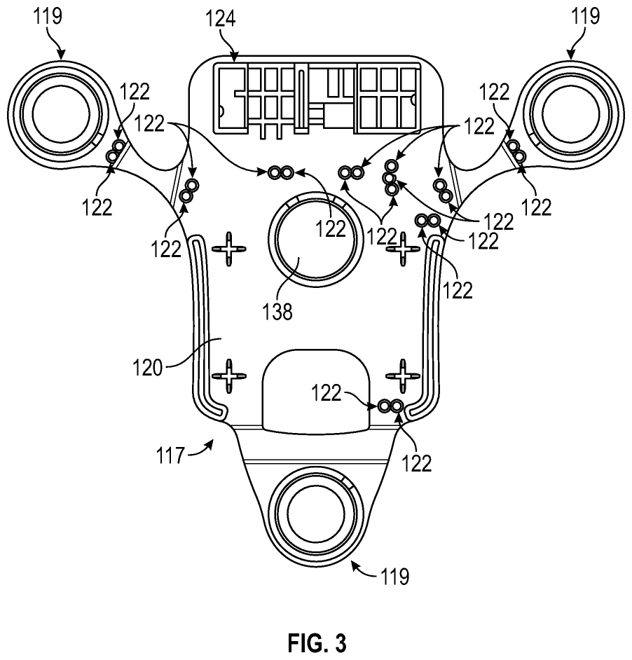

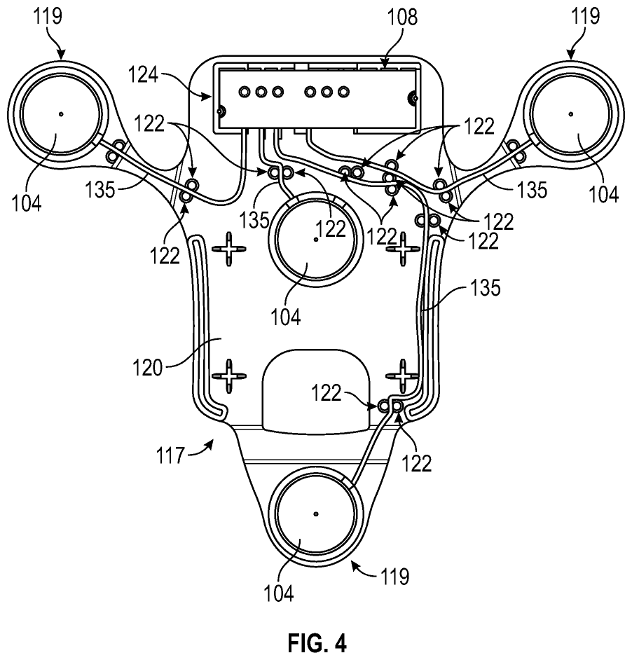

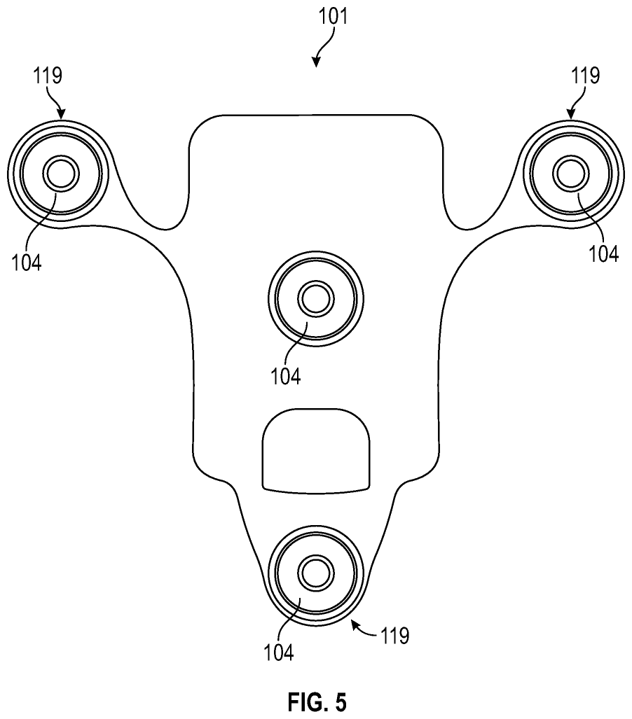

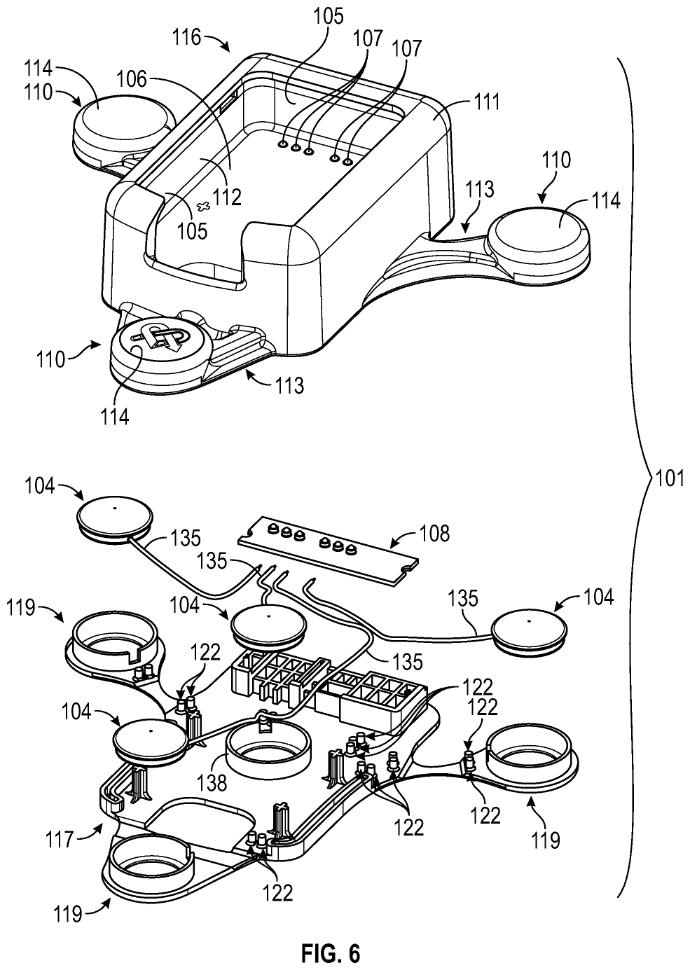

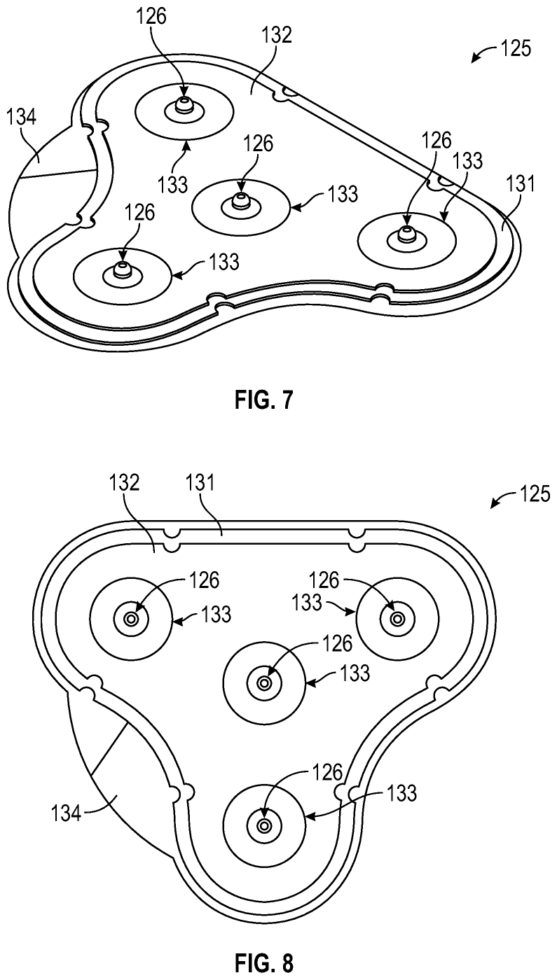

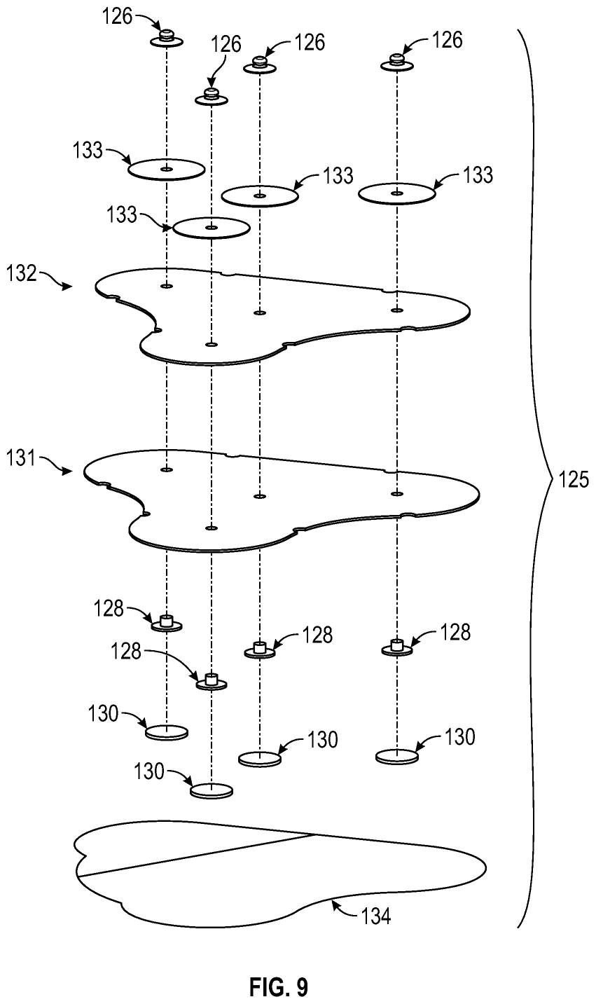

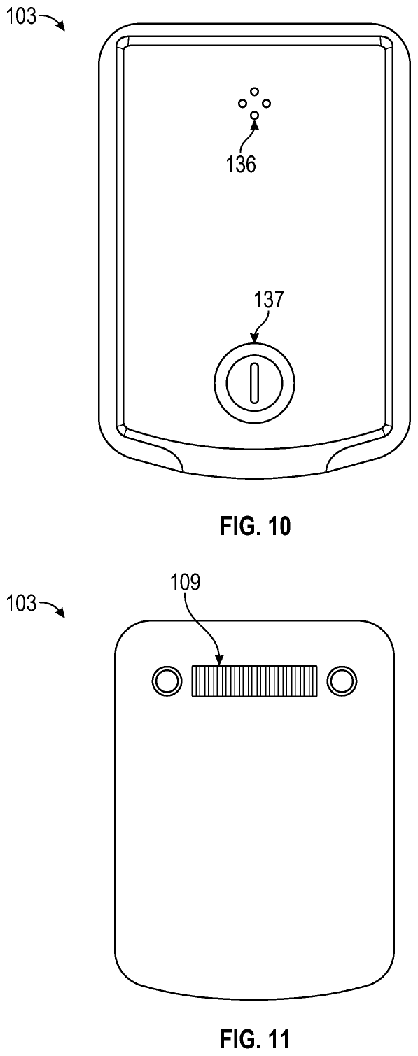

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a heart monitor system 100, which can be worn by a patient for an extended period of time and is entirely reusable with the exception of disposable electrode pads.

An embodiment of the invention, as shown and described by the various figures and accompanying text, provides a heart monitor device 100, system, and/or method capable of advantageously harvesting and monitoring a plurality of physiological signals. This device may be used in a formal medical setting (medically prescribed form), or as an over-the-counter (OTC) device available for commercial sale to the public for those interested in general health and fitness.

An embodiment of a heart monitor system 100 may include a flexible carrier member 101 and an electronic monitoring unit 103.

According to one embodiment of the heart monitor system 100, an electronic monitoring unit 103 may advantageously feature a water-resistant housing. The electronic monitoring unit 103 may include a single system on a chip (SoC), processor, microcontroller, or the like capable of executing programmable functions, including performing wireless communication via an integrated antenna.

The electrical elements of the electronic monitoring unit 103 may be configured to deliver overall device functionality. For example, and without limitation, the electronic monitoring unit 103 may be configured to carry circuitry necessary to advantageously perform biosignal harvest, evaluation, and intercommunication activities. Also, by way of example, and not as a limitation, the electrical elements of the electronic monitoring unit 103 may have the ability to harvest, record, or analyze input data channels. The electronic monitoring unit 103 may also include one or more user interface (which may include, by way of example and not as a limitation, one or more of an integrated fingertip ECG electrode, liquid crystal display (LCD), touch-screen display, indicator lights, power switch/button, recording activation button 137, and microphone 136).

The electronic monitoring unit 103 may be carried by the flexible carrier member 101. The flexible carrier member 101 may carry the electronic monitoring unit 103 and secure to a patient's body using one of at least three possible options.

In one embodiment a plurality of electrode pads 104 may secure directly to the flexible carrier member 101. In such an embodiment, the electrode pads 104 may have an adhesive surface on a first side and secure to the flexible carrier member 101 on an opposing second side. The adhesive material on the adhesive side may have sufficient strength to secure the electrode pad to the patient's body and carry the weight of the heart monitor system 100. The support may be provided by a single electrode pad 103 or by a combination or one or more electrode pads 104 in the heart monitor system 100. In such an embodiment, each of the electrode pads 104 may include a connector adapted to secure directly to a connector 115 located on the flexible carrier member 101.

In a second embodiment, a liner 125 may secure to the flexible carrier member 101 on a first side. A second, opposing side of the liner 125 may include an adhesive layer 131. In such an embodiment, the adhesive layer 131 of the liner 125 may be positioned proximate a patient's skin and the adhesive layer may adhere to the skin and support the heart monitor system 100. The second side of the liner 125 may include a plurality of connectors 126 configured to mate with corresponding connectors located on electrode pads 104 secured to the patient's body. Each of the connectors 126 may form an electrical connection between the liner 125 and the electrode pad 104 to which the connector 126 is secured. Data collected by the electrode pad 104 may be provided to the electronic monitoring unit 103 through the electrical connection formed by the connector 126. By way of example and not as a limitation, the connectors may be snaps or the like. The adhesive layer may have apertures, or be discontinuous, in locations corresponding to the placement of the connectors 126.

In a third embodiment, a liner 125 may secure to the flexible carrier member 101 on a first side. A second, opposing side of the liner 125 may include an adhesive layer 131. In such an embodiment, the adhesive layer 131 of the liner 125 may be positioned proximate a patient's skin and the adhesive layer may adhere to the skin and support the heart monitor system 100. The second side of the liner 125 may include a plurality of hydrogel deposits 130 configured to form an electrical connection with corresponding electrical contacts located on electrode pads secured to the patient's body. Each of the hydrogel deposits 130 may form an electrical connection between a connector 126 proximate the respective hydrogel deposit 130 and the electrode pad proximate the hydrogel deposit 103. Data collected by the electrode pad may be provided to the electronic monitoring unit 103 through the electrical connection formed by the hydrogel deposit 130.

Each hydrogel deposit 130 of the liner 125 may have a corresponding connector 126 located on the first side of the liner 125. Each hydrogel deposit 130 on the second side of the liner 125 may be physically located directly opposing a corresponding connector 126 on the first side of the liner 125 and be in electrical communication with the corresponding connector 126 to form an electrical connection between the two. While the hydrogel deposit 130 on the second side of the liner 125 creates an electrical connection with an electrode pad 104 worn by the patient, the connector 126 on the first side of the liner 125 creates an electrical and physical connection with the flexible carrier member 101. The connector 126 on the first side may be a male or female connector and mate with a corresponding connector on the flexible carrier member 101. The connector 126 may be a snap type connector or the like. The connector 126 on the first side of the liner 125 may secure to a mating connector 115 carried by the flexible carrier member 101 to create a physical and electrical connection between the two.

The adhesive layer 131 of the liner 125 may have a first and second, opposing side. The first side of the adhesive layer 131 may be covered with an adhesive adapted to adhere to the skin of a patient whose heart is being monitored. The second, opposing side of the adhesive layer 131 may secure to a foam layer 132 of the liner 125.

In embodiments employing a liner, the liner 125 may include a flexible, woven material. The liner 125 may be constructed from a breathable material allowing air to pass from a first side of the liner 125 to a second side of the liner 125. The liner 125 may include foam and mesh layers. The adhesive layer 131 may be a foam material. The foam layer 132 may be a foam material and have an outer perimeter smaller than the outer perimeter of the adhesive layer 131. An entirety of the foam layer 132 may be carried within the perimeter of the adhesive layer 131. An adhesive protector 134 may be secured to the first side of the adhesive layer 131 prior to securing the liner 125 to a patient.

A plurality of fasteners 126 may be carried on the first side of the liner 125. Each of the fasteners 126 may be secured to the liner using a respective securing component 128. Each of the plurality of fasteners 126 may positioned on a first side of the liner 125. A respective securing component 128 for each of the fasteners 126 may be positioned on a second side of the adhesive layer 131. Such a configuration positions the adhesive layer 131 and the foam layer 132 between respective fastener 126 and securing components 128. The respective fastener 126 and securing components 128 may secure to one another in a fixed location on the liner 125. In one embodiment, the fastener may be a socket or a stud of a snap. A washer 133, which may be of a polyester material, may be located between each fastener 126 and the foam layer 132.

The liner 125 may be configured to collectively support the entirety of the combined weight of the heart monitor system 100. The liner 125 may be configured to adhere to the patient's skin for a period of at least seven days without causing irritation or discomfort to the patient.

Each hydrogel deposit 130 may be configured to contact and form an electrical connection with an electrode adapted to monitor a patient's heart.

The flexible carrier member 101 may include a front receptacle portion 116, a back cover portion 117, and electronics component secured between and carried by the receptacle portion 116 and the back cover portion 117. The flexible carrier member 101 may be formed from silicon molded components or may include a monolithic silicon molded component.

The front receptacle portion 116 of the cradle member 101 may include a plurality of sidewalls 105 defining a central void. The sidewalls may be flexible and adapted and configured to carry an electronic monitoring unit 103 within the sidewalls with an interference fit. The central void may have an open front side, which may allow the electronic monitoring unit 103 to be inserted into or removed from the central void. The central void may have a rear portion 106 connecting to each of the sidewalls 105. The bottom sidewall 105 may be discontinuous to provide a means for inserting and removing the electronic monitoring unit 103 from the flexible carrier member 101. When the heart monitor system is worn by a patient, the rear portion 106 may be positioned between the patient and the electronic monitoring unit 103. The rear portion 106 may have one or more connector openings 107 extending through an entirety of a thickness of the rear portion 106. The one or more connector openings 107 may be configured to provide a passageway through which a connector 108, or contacts of a connector 108, carried between the back cover portion 117 and the front receptacle portion 116 may be positioned in physical contact with a connector 109, or contacts of a connector 109, carried by the electronic monitoring unit 103 when the electronic monitoring unit 103 is carried by the flexible carrier member 101.

The front receptacle portion 116 may include or be formed as a monolithic high-pressure silicon molded device designed to removably carry the electronic monitoring unit 103 within a central void, which may be located in the front receptacle portion 116 of the flexible carrier member 101. A plurality of connector covers 110 may be included in the flexible carrier member 101 and may be integrated into the silicon device forming the front receptacle portion 116. A flexible carrier member 101 for use with a dual channel electronic monitoring unit 103 may include three or four connector covers 110. A flexible carrier member 101 for use with a single channel electronic monitoring unit 103 may include two, three or four connector covers 110. Each of the connector covers 110 may carry a connector 104 aligned with a connector 126 carried by the liner 125. Each connector 104 of the flexible carrier member 101 may be adapted to secure to a connector 126 of the liner 125 and form a mechanical and electrical connection therebetween. The electrical signal detected by an electrode secured to a patient may be provided to the electronics monitoring unit 103 by traveling through the hydrogel deposit 130, securing component 128, connector 126, connector 104, lead 135, connector 108, and then being delivered to connector 109.

In one embodiment having three connector covers 110 a first connector cover 110 may extend outwardly of a sidewall 105 defining a left side of the central void and a second connector cover 110 may extend outwardly of a sidewall 105 defining a right side of the central void. In such an embodiment, the first and second connector covers 110 may have midpoints located equal distances below, above, or aligned with the sidewall 105. A third connector cover 110 may be located below both the first and second connector covers 110 and extending downwardly from the sidewall 105 defining the bottom side of the central void. The first, second, and third connector covers 110 may be configured to form an isosceles triangle with angles located at the midpoints of each of the connector covers 110. The distance between the first and third connector covers 110 may be equal to the distance between the second and third connector covers 110.

In an embodiment having three connector covers 110, a first connector cover 110 may extend upwardly from a sidewall 105 defining a left side of the central void and a second connector cover 110 may extend upwardly from a sidewall 105 defining a right side of the central void. The third connector cover 110 may extend away from the flexible carrier member 101 proximate a gap in the sidewall 105 and may extend downwardly from the sidewall 105. In such an embodiment, the first and second connector covers 110 may have midpoints located equal horizontal distances to the left or right, respectively, of the sidewall 105 defining the left or right side of the central void. A third connector cover 110 may extend downwardly from a bottom sidewall 105. The midpoint of the third connector cover 110 may below the bottom sidewall 105.

In each embodiment of the flexible carrier member 101, each of the connector covers 110 may extend orthogonally away from a sidewall or may extend away from a sidewall at an angle other than 90 degrees. Each of the connector covers 110 may be positioned to maintain at least 10 mm between the midpoints of each connector cover 110 in each electrode vector pair.

The bottom sidewall 105 of the receptacle portion 116 of the cradle member 101 may include a gap, which may create a bottom left sidewall 105 and a bottom right sidewall 105. The sidewall 105 gap may provide access to one or more connectors of the electronic monitoring unit 103 while the electronic monitoring unit 103 is carried by the cradle member 101. The accessible connectors may provide access to, by way of example and not as a limitation, power or data ports of the electronic monitoring unit 103.

The front receptacle portion 116 of the cradle member 101 may include a continuous sidewall 105 defining a central aperture with the sidewall 105 including a gap along a portion of one side of the central aperture. In one embodiment, this gap may be along a bottom of the central aperture. Each sidewall may have an upright portion 112 extending essentially orthogonally from the rear portion 106 of the receptacle portion 116 of the cradle member 101. A first end of the upright portion 112 may be secured to or integrated with the rear portion 106 and a second end of the upright portion 112 may be secured to or integrated with a bevel portion 111. An inner side of the bevel portion 111 may define an inner perimeter, which is smaller than an inner perimeter defined by an inner side of the upright portion 112. The inner side of the bevel portion 111 may extend over an outer perimeter of the electronic monitoring unit 103 when the electronic monitoring unit 103 is positioned in the central void of the cradle member 101 and retain the electronic monitoring unit 103 within the flexible carrier member 101.

Each connector cover may include a lead cover portion 113 and a sensor cover portion 114. The lead cover portion 113 may secure directly to a sidewall 105 or the rear portion 106 of the front receptacle portion 116 of the flexible carrier member 101 and to the sensor cover portion 114. The lead cover portion 113 may be configured to receive a lead 135 creating a physical and electrical connection between a connector 104 and a connector 108 of the flexible carrier member 101. The sensor cover portion 114 may be configured to receive or cover a connector 104.

A plurality of leads 135, connectors 104, and a connector 108 may be carried within the flexible carrier member 101, between the front receptacle portion 116 and the back cover portion 117, and provide electrical signals to the electronic monitoring unit 103. A separate lead 135 may connect each of the connectors 104 to a respective contact on the connector 108. Each of the connectors 104 may be configured to contact a connector 126 and create an electronic connection with a hydrogel deposit 130 on the liner 125.

The back cover portion 117 of the flexible carrier member 101 may be cooperatively configured with the front receptacle portion 116 of the cradle member 101 to allow the two portions 117, 116 to mate and contain the electronics components, including, but not limited to, the leads 135, connectors 104, and connector 108, between the portions 117, 116 with a hermetic seal. The back cover portion 117 may have a plurality of connector receptacles 119, each of which may correspond to a connector cover 110 of the receptacle portion 116. One or more connector covers 110 of the front receptacle portion 116 may have an inner diameter adapted to receive an outer diameter of a connector receptacle 119 of the back cover portion 117. The connector cover 110 of the front receptacle portion 116 may secure to the connector receptacle 119 of the back cover portion 117 with an adhesive, interference fit, or the like. The back cover portion 117 may have an inner side 120 and an opposing outer side. When worn by a patient, the outer side may be proximate the patient's skin. The inner side 120 of the back cover portion 117 may have channels, recesses, protuberances, or the like, which may be used to retain, route, or otherwise position one or more leads 135. Channels, protuberances, or like structures may be located on the inner side 120 of the back cover portion 120 to provide structural support or rigidity for the back cover portion 117. Such channels, protuberances, or the like may be positioned and sized to contact the inner side of the front receptacle portion 116 of the flexible carrier member 101 when the front receptacle portion 116 and back cover portion 117 are mated. Channels, protuberances, or like structures on the inner side 120 of the back cover portion 120 to provide rigidity to the back cover portion 120 may not contact the inner side of the front receptacle portion 116.

One or more lead clips 122 may be located on the inner side 120 of the back cover portion 117. Each lead clip 122 may include two protuberances extending away from the inner side 120 of the back cover portion 117 and cooperatively configured to retain a lead 135 therebetween. The lead 135 may be secured between the protuberances of the lead clip 122 with an interference fit, adhesive, or other securing means. The lead clips 122 may be positioned along the inner side 120 of the back cover portion 117 to route the lead 135 between the connector 108 and the connector 104. The lead clips 122 may secure the lead 135 to the back cover portion 117 and provide strain relief to the lead 135. In one embodiment, the lead clips 122 may be located on an inner side of the receptacle portion 116 in addition to or in lieu of lead clips 122 located on the inner side 120 of the back cover portion 117.

The inner side 120 of the back cover portion 117 may include a raised ridge 124 corresponding to an outer diameter of the connector 108. The raised ridge 124 may be sized and positioned to extend slightly beyond the outer perimeter of the connector 108. The connector 108 may be positioned within a perimeter defined by the raised ridge 124. The raised ridge 124 may fully enclose and define a perimeter or the raised ridge 124 or may partially define a perimeter inside of which the connector 108 may be positioned. The connector 108 may be secured within the raised ridge 124 with an interference fit, adhesive, or other securing means.

One embodiment may include a connector 104 or dry electrode carried within the perimeter of the back cover portion 117 without a corresponding connector cover 110. In such an embodiment, the back cover portion 117 may include an aperture 138 through an entirety of a thickness of a surface. The aperture 138 may be sized to receive and carry a connector 104 or a dry electrode. A lead 135 may connect this connector 104 or dry electrode to the connector 108. This fourth connector 104 or dry electrode may be positioned equidistant from both the first and second connectors 104. In embodiments with a dry electrode rather than a connector 104 positioned in aperture 138, the liner 125 may have a corresponding aperture, rather than a connector. The aperture in the liner 125 may be adapted to allow the dry electrode carried by the flexible carrier member 101 to make contact directly with the skin of the patient.

By way of example and without limitation the connector 104 and corresponding connector covers 110, connector receptacles 119, or aperture 138 in the back cover portion 117, may be designed to position and carry the connectors 104 as one of a single channel configuration or a dual channel configuration. The single channel configuration may support right arm RA and left leg LL with an approximate distance between electrodes of 8 centimeters, which may advantageously allow capture of a clean signal with enough amplitude of the electrical movement. The single channel configuration may also support a third electrode that may be used as Right Leg RL drive or reference electrode. The third electrode may be a dry electrode. The two channel configuration may support right arm RA, left leg LL, right leg RL, left arm LA and a reference electrode. The reference electrode may be a dry electrode. The spacing for standard ECG lead II (right arm RA and left leg LL) may be 8 CM at its approximate distance. The second ECG channel (right leg RL, left arm LA, and reference electrode) may be between 4 cm and 8 cm apart. By way of example and without limitation, these requirements may advantageously be supported using four electrodes, with the fourth electrode being interchangeable for the Right Leg drive and reference electrode.

Thoughtful selection of electrode pad diameter may advantageously provide a signal to noise ratio needed for clean ECG while still adhering to the body for seven days. For example, and without limitation, standard electrodes may be characterized by a diameter of approximately 1.5 cm.

By way of example and without limitation, the heart monitor system 100 may be made available in either a medically-prescribed form or as an over-the counter (OTC) device. In the prescribed form, the heart monitor system 100 may use one or more communication means to send the acquired data to a monitoring center. By way of example and without limitation, the monitoring center may feature human over read, or no human over read (e.g., fully automated monitoring). By way of example and without limitation, the monitoring center may analyze and process the data through a proprietary software system and process, and reports may be generated and sent to a physician, or the data may be sent directly to the physician in raw form and/or as an automatically configured report. The information may also be transmitted to a mobile device application which may be downloaded by a user onto her own instance of the heart monitor system software.

In another embodiment, the heart monitor system 100 may employ its means of communication to advantageously display acquired information to a user with a recreational purpose. By way of example and without limitation, an interface may be provided by a computer program or mobile device application, for which the heart monitor system may be configured to recognize a user's selected interfacing means and may transmit the acquired physiological data according to the interface detected. The software program/application may be configured to read data sent to it by the heart monitor system and display those data to the user in such a way that it is meaningful and easy to understand. This application may generally have advantageous uses in the realm of individual health and fitness. The interfacing program/application may also be configured to advantageously generate warnings and reports to inform the user of any potential health problems detected by the heart monitor system. All versions of the interface may be capable of providing different customizable reports of both physiological events and trending. Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. A system for heart monitoring comprising:
an electronic monitoring unit;
a flexible carrier member comprising:
a central void adapted to carry the electronic monitoring unit within the void, and
a back surface having a first connector component; and
a liner comprising:
a back, adhesive surface,
an opposing front surface, and
a second connector component located on the front surface and adapted to secure to the first connector component of the flexible carrier member.

2. The system of claim 1 wherein the first connector component includes a socket or stud of a snap fastener and the second connector component includes a socket or stud of a snap fastener.

3. The system of claim 1 wherein the electronic monitoring unit further comprise a microphone.

4. The system of claim 3 wherein the electronic monitoring unit activates the microphone to capture an audio recording and the audio recording is associated with a time period of recording activity.

5. The system of claim 1 wherein the second connector is adapted to be electrical and physical communication with the first connector component.

6. The system of claim 1 wherein the liner further comprises:
a foam layer having a front and an opposing, rear surface, wherein the front surface of the foam layer is the front surface of the liner; and
an adhesive layer having a front and an opposing, back surface, wherein the back surface of the adhesive layer is the back, adhesive surface of the liner and the rear surface of the foam layer secures to the front surface of the adhesive layer.

7. The system of claim 6 further comprising:
a securing component positioned on the back surface of the adhesive layer and in electrical communication with the second connector, wherein the second connector is positioned on the front surface of the foam layer.

8. The system of claim 7 further comprising a hydrogel deposit in electrical communication with the securing component.

9. The system of claim 8 further comprising a washer captured between the second connector and the foam layer.

10. The system of claim 6 wherein an outer perimeter of the foam layer is contained within an outer perimeter of the adhesive layer.

11. The system of claim 1 wherein the flexible carrier member further comprises:
a front receptacle portion having the central void; and
a back cover portion; and
wherein the first connector component is carried between the front receptacle portion and the back cover portion.

12. The system of claim 11 wherein the front receptacle portion further comprises:
a plurality of sidewalls defining the central void;
a lead cover portion extending from one of the plurality of sidewalls distal the central void having a first end secured to the one of the plurality of sidewalls; and
a connector cover secured to a second end of the lead cover portion.

13. The system of claim 11 wherein the back cover portion comprises:
an aperture adapted to carry the first connector component.

14. The system of claim 13 wherein the aperture, the first connector component, and the second connector component are in vertical alignment.

15. The system of claim 11 wherein the back cover portion comprises:
a connector receptacle adapted to carry the first connector component.

16. The system of claim 12 further comprising:
an aperture located through an entirety of a thickness of the front receptacle portion;
a connector secured between the front receptacle portion and the back cover portion having at least one contact accessible through the aperture; and
a lead located between the lead cover portion and the back cover portion and having a first end secured to the first connector component and a second end secured to the connector.

17. A system for heart monitoring comprising:
an electronic monitoring unit;
a flexible carrier member comprising:
a front receptacle portion having a plurality of sidewalls defining a central void adapted to carry the electronic monitoring unit within the void,
an connector aperture located through an entirety of a thickness of the front receptacle portion,
a back cover portion having a back surface,
a connector secured between the front receptacle portion and the back cover portion having at least one contact accessible through the connector aperture,
a first connector component carried between the front receptacle portion and the back cover portion,
a lead cover portion extending from one of the plurality of sidewalls distal the central void having a first end secured to the one of the plurality of sidewalls,
a connector cover secured to a second end of the lead cover portion, and
a lead located between the lead cover portion and the back cover portion and having a first end secured to the first connector component and a second end secured to the connector;
a liner comprising:
an adhesive layer having a front surface, and an opposing back, adhesive surface,
a securing component positioned on the back surface of the adhesive layer,
a second connector,
a foam layer having a rear surface, secured to the front surface of the adhesive layer, and a front surface,
a hydrogel deposit in electrical communication with the securing component, and
a washer captured between the second connector and the foam layer,
wherein the second connector is adapted to physically secure to the first connector component of the flexible carrier member and create an electrical connection therebetween,
wherein the securing component is in electrical communication with the second connector, and
wherein an outer perimeter of the foam layer is contained within an outer perimeter of the adhesive layer.

18. The system of claim 17 wherein the back cover portion comprises:
an aperture adapted to carry the first connector component in vertical alignment with the second connector component.

19. A system for heart monitoring comprising:
an electronic monitoring unit;
a flexible carrier member comprising:
a front receptacle portion having a plurality of sidewalls defining a central void adapted to carry the electronic monitoring unit within the void,
an connector aperture located through an entirety of a thickness of the front receptacle portion,
a back cover portion having a back surface with an aperture,
a connector secured between the front receptacle portion and the back cover portion having at least one contact accessible through the connector aperture,
a first carrier connector component carried between the front receptacle portion and the back cover portion,
a second carrier connector component carried between the front receptacle portion and the aperture of the back cover portion,
a lead cover portion extending from one of the plurality of sidewalls distal the central void having a first end secured to the one of the plurality of sidewalls,
a connector cover secured to a second end of the lead cover portion positioned to cover the first carrier connector component, and
a lead located between the lead cover portion and the back cover portion and having a first end secured to the first carrier connector component and a second end secured to the connector;
a liner comprising:
an adhesive layer having a front surface, and an opposing back, adhesive surface,
a securing component positioned on the back surface of the adhesive layer,
a foam layer having a rear surface, secured to the front surface of the adhesive layer, and a front surface,
a hydrogel deposit in electrical communication with the securing component, and
a first liner connector positioned on the front surface of the foam layer,
a second liner connector positioned on the front surface of the foam layer,
a washer captured between the first liner connector and the foam layer,
wherein the first liner connector is adapted to physically secure to the first carrier connector component of the flexible carrier member and create an electrical connection therebetween,
wherein the second liner connector is adapted to physically secure to the second carrier connector component of the flexible carrier member and create an electrical connection therebetween,
wherein the securing component is in electrical communication with the first liner connector, and
wherein an outer perimeter of the foam layer is contained within an outer perimeter of the adhesive layer.

20. The system of claim 19 wherein
the flexible carrier member further comprises:
a third carrier connector component carried between the front receptacle portion and the back cover portion,
a second lead cover portion extending from one of the plurality of sidewalls distal the central void having a first end secured to the one of the plurality of sidewalls,
a second connector cover secured to a second end of the second lead cover portion positioned to cover the third carrier connector component,
a second lead located between the second lead cover portion and the back cover portion and having a first end secured to the third carrier connector component and a second end secured to the connector,
a fourth carrier connector component carried between the front receptacle portion and the back cover portion,
a third lead cover portion extending from one of the plurality of sidewalls distal the central void having a first end secured to the one of the plurality of sidewalls,
a third connector cover secured to a second end of the third lead cover portion positioned to cover the fourth carrier connector component, and
a third lead located between the third lead cover portion and the back cover portion and having a first end secured to the fourth carrier connector component and a second end secured to the connector,
wherein the liner further comprises:
a third liner connector positioned on the front surface of the foam layer, and
a fourth liner connector positioned on the front surface of the foam layer,
wherein the third liner connector is adapted to physically secure to the third carrier connector component of the flexible carrier member and create an electrical connection therebetween, and
wherein the fourth liner connector is adapted to physically secure to the fourth carrier connector component of the flexible carrier member and create an electrical connection therebetween.
